(19) United States
(12) Patent Application Publication
(10) Pub. No.: US 2013/0156855 A1
(43) Pub. Date: Jun. 20, 2013

(54) ACNE TREATMENT

(71) Applicant: Aviv Ben-Menachem, New York, NY (US)

(72) Inventor: Aviv Ben-Menachem, New York, NY (US)

(21) Appl. No.: 13/328,833

(22) Filed: Dec. 16, 2011

(51) Int. Cl. A61K 31/205; A61K 31/525; A61K 31/455; A61K 31/675; A61P 17/10; A61K 31/714; A61K 31/4188; A61K 9/00; A61K 9/28; A61K 31/51; A61K 31/519

(52) U.S. Cl. 424/474; 514/556; 514/276; 514/251; 514/356; 514/89; 514/249; 514/52; 514/387; 424/400

(57) ABSTRACT

One example embodiment includes a chemical composition for the treatment of acne. The chemical composition includes pantothenic acid and L-Carnitine.

### CROSS-REFERENCE TO RELATED APPLICATIONS

Not applicable.

### BACKGROUND OF THE INVENTION

Acne is a condition that affects many individuals. Acne can be especially common in adolescence, but may persist throughout an individual's lifetime. Most treatments focus on treating the actual outbreak of acne. That is, once the acne begins to appear, the individual spreads a topical treatment on his/her skin at the location of the outbreak.

However, this method of treatment has a number of drawbacks. For example, the individual must spend a significant amount of time looking for outbreaks. I.e., the individual must inspect his/her skin multiple times per day. This often causes the individual to focus on any minor flaws and can damage the self-esteem of the individual. In addition, the individual must be diligent otherwise outbreaks may go untreated. Finally, the individual is treating the result, rather than the outbreak.

In addition, many treatments include harsh chemicals. These chemicals are often intended to kill or break down any tissue in a localized area. Thus, the amount used must be carefully controlled to ensure that the individual does not misuse or overuse the treatment. In addition, these chemicals can cause their own health problems, even if used correctly.

Accordingly, there is a need in the art for an acne treatment that treats the underlying cause of acne. In addition, there is a need in the art for the treatment that does not include harsh chemicals. Further, there is a need in the art for the treatment to include natural ingredients.

### BRIEF SUMMARY OF SOME EXAMPLE EMBODIMENTS

This Summary is provided to introduce a selection of concepts in a simplified form that are further described below in the Detailed Description. This Summary is not intended to identify key features or essential characteristics of the claimed subject matter, nor is it intended to be used as an aid in determining the scope of the claimed subject matter.

One example embodiment includes a chemical composition for the treatment of acne. The chemical composition includes pantothenic acid and L-Carnitine.

One example embodiment includes a chemical composition for the treatment of acne. The chemical composition includes pantothenic acid and L-Carnitine. The chemical composition also includes one or more additional vitamins.

One example embodiment includes a chemical composition for the treatment of acne. The chemical composition includes pantothenic acid and L-Carnitine. The chemical composition also includes one or more additional vitamins and one or more chemical stabilizers. The chemical composition further includes an enteric coating.

These and other objects and features of the present invention will become more fully apparent from the following description and appended claims, or may be learned by the practice of the invention as set forth hereinafter.

### DETAILED DESCRIPTION OF SOME EXAMPLE EMBODIMENTS

Reference will now be made to the figures wherein like structures will be provided with like reference designations. It is understood that the figures are diagrammatic and schematic representations of some embodiments of the invention, and are not limiting of the present invention, nor are they necessarily drawn to scale.

The invention can include a chemical composition effective in the treatment of acne. The chemical composition can be a nutritional supplement, such as a pill, gel or paste, ingested by the user for acne treatment. Acne is a common human skin disease, characterized by areas of skin with seborrhea (scaly red skin), comedones (blackheads and whiteheads), papules (pinheads), pustules (pimples), Nodules (large papules) and possibly scarring.

In at least one implementation, the chemical composition can include pantothenic acid (aka pantothenate or Vitamin B5). Pantothenic acid has a chemical formula of C₉H₁₇NO₅ and has an IUPAC name of 3-[(2,4-dihydroxy-3,3-dimethylbutanoyl)amino]propanoic acid. The percentage, by weight, of pantothenic acid can be between 17 percent and 53 percent. For example, the percentage, by weight of pantothenic acid can be approximately 35%. The daily dosage of pantothenic acid can be between 1100 mg and 3300 mg. For example, the daily dosage of pantothenic acid can be approximately 2200 mg. As used in the specification and the claims, the term approximately shall mean that the value is within 10% of the stated value, unless otherwise specified.

In at least one implementation, within the human body pantothenic acid is used in, and increases the rate of, the synthesis of coenzyme A (CoA—chemical formula C₂₁H₃₆N₇O₁₆P₃S). Coenzyme A may act as an acyl group carrier to form acetyl-CoA and other related compounds; this is a way to transport carbon atoms within the cell. CoA is important in energy metabolism for pyruvate to enter the tricarboxylic acid cycle (TCA cycle) as acetyl-CoA, and for a-ketoglutarate to be transformed to succinyl-CoA in the cycle. CoA is also important in the biosynthesis of many important compounds such as fatty acids, cholesterol, and acetylcholine. CoA is incidentally also required in the formation of ACP, which is also required for fatty acid synthesis in addition to CoA.

In high quantities, COA can increase the rate at which the oily sebum in the skin is broken down. I.e., CoA prevents the skin from becoming greasy or oily. This, in turn, reduces the occurrence of acne. That is, CoA helps the body to maintain a balanced skin composition.

In at least one implementation, the chemical composition can also include L-Carnitine. Carnitine exists in two stereoisomers: Its biologically active form is L-carnitine, whereas its enantiomer, D-carnitine, is biologically inactive. Carnitine has a chemical formula of C₇H₁₅NO₃ and has an IUPAC name of 3-hydroxy-4-(trimethylazaniumyl)butanoate. Carnitine is a quaternary ammonium compound biosynthesized from the amino acids lysine and methionine. In living cells, it is required for the transport of fatty acids from the cytosol into the mitochondria during the breakdown of lipids (fats) for the generation of metabolic energy. The percentage, by weight, of L-Carnitine can be between 6 percent and 18 percent. For example, the percentage, by weight of L-Carnitine can be approximately 12 percent. The daily dosage of L-Carnitine can be between 365 mg and 1100 mg. For example, the daily dosage of L-Carnitine can be approximately 733 mg.

In at least one implementation, the chemical composition can further include other vitamins. For example, Table 1 shows additional vitamins that can be include as a percentage of weight.

TABLE 1

| | Range Percentage | | Approximate |
|---|---|---|---|
| Additional | by Weight | | Percentage |
| Vitamins | Low | High | By Weight |
| Thiamin | 0.0478% | 0.1435% | 0.0956% |
| Riboflavin | 0.0542% | 0.1626% | 0.1084% |
| Niacin | 0.6376% | 1.9129% | 1.2753% |
| Vitamin B6 | 0.0638% | 0.1913% | 0.1275% |
| Folate | 0.0128% | 0.0383% | 0.0255% |
| Vitamin B12 | 0.0002% | 0.0006% | 0.0004% |
| Biotin | 0.0096% | 0.0287% | 0.0191% |

Table 2 shows additional vitamins as a daily dosage.

TABLE 2

| | | | | | Approximate | | % Daily Value |
|---|---|---|---|---|---|---|---|
| Additional | Range Daily Dosage | | | | Daily | | of Approximate |
| Vitamins | Low | | High | | Dosage | | Daily Dosage |
| Thiamin | .78 | mg | 2.25 | mg | 1.5 | mg | 100 |
| Riboflavin | 0.85 | mg | 2.55 | mg | 1.7 | mg | 100 |
| Niacin | 10 | mg | 30 | mg | 20 | mg | 100 |
| Vitamin B6 | 1 | mg | 3 | mg | 2 | mg | 100 |
| Folate | 200 | mcg | 600 | mcg | 400 | mcg | 100 |
| Vitamin B12 | 3 | mcg | 9 | mcg | 6 | mcg | 100 |
| Biotin | 150 | mcg | 450 | mcg | 300 | mcg | 100 |

In at least one implementation, the chemical composition can additionally include stabilizers. In chemistry a stabilizer is a chemical which tends to inhibit the reaction between two or more other chemicals. It can be thought of as the antonym to a catalyst. The term can also refer to a chemical that inhibits separation of suspensions, emulsions, and foams. For example, stabilizers can include dicalcium phosphate, microcrystialline cellulose, croscarmellose sodium, stearic acid, silica, Magnesium stearate or any other desired stabilizers.

In at least one implementation, the chemical composition can also include an enteric coating. An enteric coating is a barrier applied to oral medication that controls the location in the digestive system, where it is absorbed. Enteric refers to the small intestine, therefore enteric coatings prevent release of medication before it reaches the small intestine. For example, the enteric coating can include polyvinyl alcohol, titantium doxide, macrogol, talc or any other desired coating materials.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. A chemical composition for the treatment of acne, the composition comprising:
pantothenic acid;
L-Carnitine; and
vitamins, wherein the vitamins include:
thiamin, wherein the percentage, by weight, of thiamin is approximately 0.1 percent;
riboflavin, wherein the percentage, by weight, of riboflavin is approximately 0.1 percent;
niacin, wherein the percentage, by weight, of niacin is approximately 1.3 percent;
vitamin B6, wherein the percentage, by weight, of vitamin B6 is approximately 0.13 percent;
folate, wherein the percentage, by weight, of folate is approximately 0.03 percent;
vitamin B12, wherein the percentage, by weight, of vitamin B12 is approximately 0.0004 percent; and
biotin, wherein the percentage, by weight, of biotin is approximately 0.02 percent.

2. The chemical composition of claim 1, wherein the percentage, by weight of the pantothenic acid is between 17 percent and 53 percent.

3. The chemical composition of claim 2, wherein the percentage, by weight of the pantothenic acid is approximately 35 percent.

4. The chemical composition of claim 1, wherein the daily dosage of the pantothenic acid is between 1100 mg and 3300 mg.

5. The chemical composition of claim 4, wherein the daily dosage of the pantothenic acid is approximately 2200 mg.

6. The chemical composition of claim 1, wherein the percentage, by weight of the L-Carnitine is between 6 percent and 18 percent.

7. The chemical composition of claim 6, wherein the percentage, by weight of the L-Carnitine is approximately 12 percent.

8. The chemical composition of claim 1, wherein the daily dosage of the L-Carnitine is between 365 mg and 1100 mg.

9. The chemical composition of claim 8, wherein the daily dosage of the L-Carnitine is approximately 733 mg.

10. A chemical composition for the treatment of acne, the composition comprising:
pantothenic acid;
L-Carnitine; and
vitamins, wherein the vitamins include:
thiamin, wherein the daily dosage of thiamin is approximately 1.5 mg;
riboflavin, wherein the daily dosage of riboflavin is approximately 1.7 mg;
niacin, wherein the daily dosage of niacin is approximately 20 mg;
vitamin B6, wherein the daily dosage of vitamin B6 is approximately 2 mg;
folate, wherein the daily dosage of folate is approximately 400 mcg;
vitamin B12, wherein the daily dosage of vitamin B12 is approximately 6 mcg; and
biotin, wherein the daily dosage of biotin is approximately 300 mg.

11.-15. (canceled)

16. A chemical composition for the treatment of acne, the composition comprising:
pantothenic acid;
L-Carnitine;
vitamins, wherein the vitamins include:
thiamin, wherein the daily dosage of thiamin is approximately 1.5 mg;
riboflavin, wherein the daily dosage of riboflavin is approximately 1.7 mg;
niacin, wherein the daily dosage of niacin is approximately 20 mg;
vitamin B6, wherein the daily dosage of vitamin B6 is approximately 2 mg;
folate, wherein the daily dosage of folate is approximately 400 mcg;
vitamin B12, wherein the daily dosage of vitamin B12 is approximately 6 mcg; and
biotin, wherein the daily dosage of biotin is approximately 300 mg;
one or more chemical stabilizers; and
an enteric coating.

17. The chemical composition of claim 16, wherein the one or more chemical stabilizers include at least one of:
dicalcium phosphate;
microcrystalline cellulose;
croscarmellose sodium;
stearic acid;
silica; or
magnesium stearate.

18. The chemical composition of claim 16, wherein the enteric coating includes:
polyvinyl alcohol;
titanium doxide;
macrogol; and
talc.

19. The chemical composition of claim 16, as a pill.

20. The chemical composition of claim 10, wherein the percentage, by weight of the pantothenic acid is between 17 percent and 53 percent.

21. The chemical composition of claim 20, wherein the percentage, by weight of the pantothenic acid is approximately 35 percent.

22. The chemical composition of claim 10, wherein the daily dosage of the pantothenic acid is between 1100 mg and 3300 mg.

23. The chemical composition of claim 22, wherein the daily dosage of the pantothenic acid is approximately 2200 mg.

24. The chemical composition of claim 10, wherein the percentage, by weight of the L-Carnitine is between 6 percent and 18 percent.

25. The chemical composition of claim 10, wherein the daily dosage of the L-Carnitine is between 365 mg and 1100 mg.
